(19) United States
(12) Patent Application Publication
(10) Pub. No.: US 2026/0284307 A1
(43) Pub. Date: Sep. 24, 2026

(54) PUMP CASSETTE WITH PULSATION ABATEMENT

(71) Applicant: Arthrex, Inc., Naples, FL (US)

(72) Inventors: Roberto C. del Cid, Naples, FL (US); James Tebo, Naples, FL (US); Ron Seese, Naples, FL (US); Christopher Maricle, Naples, FL (US); Christopher Lynn, Austin, TX (US)

(73) Assignee: Arthrex, Inc., Naples, FL (US)

(21) Appl. No.: 19/570,824

(22) Filed: Mar. 18, 2026

Related U.S. Application Data
(60) Provisional application No. 63/773,649, filed on Mar. 18, 2025.
(60) Provisional application No. 63/883,731, filed on Sep. 18, 2025.

(51) Int. Cl. A61M 5/168; A61M 5/14

(52) U.S. Cl. CPC A61M 5/16854; A61M 5/1413; A61M 2205/3331

(57) ABSTRACT

A pump cassette includes a cassette housing defining an input aperture configured to receive a surgical solution and an output aperture configured to expel the surgical solution. A flow path is defined between the input aperture and the output aperture and includes a sensor segment having a sensor segment input opening, a sensor segment output opening, and a pressure module configured to react to changes in pressure. A sensor segment wall defines a portion of the flow path between the sensor segment input opening and the sensor segment output opening. The sensor segment wall includes a first sensor segment wall portion located between the sensor segment input opening and the sensor segment output opening and a second sensor wall portion extending transversely from the first sensor segment wall portion to a sensor segment wall terminal end.

### CROSS-REFERENCE TO RELATED APPLICATIONS

This application claims priority under 35 U.S.C. § 119(e) and the benefit of U.S. Provisional Application No. 63/773,649 entitled PUMP CASSETTE WITH PULSATION ABATEMENT, filed on Mar. 18, 2025, and U.S. Provisional Application No. 63/883,731 entitled PUMP CASSETTE WITH PULSATION ABATEMENT, filed on Sep. 18, 2025, the entire disclosures of which are incorporated herein by reference.

### BACKGROUND

The present disclosure generally relates to a pump cassette utilized during a medical procedure to transfer a surgical solution relative to a region of a patient's body. More particularly, the pump cassette includes various features for optimizing the flow rate of the surgical solution. Traditionally, surgical pumps have been implemented to deliver cleaning solutions. In various implementations, the disclosure may provide a pump cassette that improves flow rate control by limiting pulses that may be associated with the operation of surgical pumps.

### SUMMARY

The disclosure generally provides for a pump cassette for a surgical pump assembly, the surgical pump assembly, and a control system of the surgical pump assembly. In various aspects, the disclosed apparatuses may be used to accomplish the transfer of surgical solution at constant, near constant, and/or stabilized flow rates that may be at near laminar flow, by limiting fluctuations in fluid transfer, and preventing air bubble (e.g., cavitation) formation. Additionally, in some implementations, the control system may monitor (e.g., sample, detect, and/or otherwise obtain) the flow rate with improved accuracy to control the pressure associated with the operation of the pump assembly. As described in detail in the following description, the pump cassette may form a tortuous flow path with one or more pressure modules, pulsation abatement components, and/or flexible membranes that may be implemented independently or in various combinations to improve the operation of the surgical pump assembly for various procedures. For example, the surgical pump assembly may include a sensor for determining a pressure within the flow path and a control system for optimizing the flow rate of the surgical solution.

In various implementations, the disclosure may provide for a pump cassette with pressure sensing. The pump cassette may include a cassette housing having a first major wall and a second major wall spaced from the first major wall by a sidewall. An input aperture may be defined by the cassette housing and configured to receive a surgical solution during a medical procedure. An output aperture may be defined by the cassette housing and configured to expel the surgical solution during the medical procedure. A flow path may be defined between the input aperture and the output aperture. The flow path may include a sensor segment that includes a sensor segment input opening, a sensor segment output opening, and a pressure module configured to react to changes in pressure. A sensor segment wall may define a portion of the flow path between the sensor segment input opening and the sensor segment output opening. The sensor segment wall may include a first sensor segment wall portion located between the sensor segment input opening and the sensor segment output opening and a second sensor wall portion extending transversely from the first sensor segment wall portion to a sensor segment wall terminal end. The flow path may follow the sensor segment wall from the sensor segment input opening past the sensor segment wall terminal end and to the sensor segment output opening.

In some implementations, the disclosure may provide for a pump cassette with pulsation abatement. The pump cassette may include a cassette housing having a first major wall and a second major wall spaced from the first major wall by a sidewall. The sidewall may include a first lateral sidewall section and a second lateral sidewall section spaced by a first longitudinal sidewall section and a second longitudinal sidewall section. An input aperture may be defined by the cassette housing and configured to receive a surgical solution during a medical procedure. An output aperture may be defined by the cassette housing and configured to expel the surgical solution during the medical procedure. A flow path may be defined between the input aperture and the output aperture. The flow path may include an input flow path segment extending from the input aperture and at least partially along the first lateral sidewall section to the second longitudinal sidewall section. The flow path may further include an output flow path segment extending at least partially along the second lateral sidewall section to the output aperture. A flow path branch may be between the input aperture and the output aperture that extends to a cavity within the cassette housing for pulse abatement of a surgical solution.

In various implementations, the disclosure may provide for a pump cassette with pressure sensing. The pump cassette may include a cassette housing having a first major wall and a second major wall spaced from the first major wall by a sidewall. An input aperture may be defined by the cassette housing and configured to receive a surgical solution during a medical procedure. An output aperture may be defined by the cassette housing and configured to expel the surgical solution during the medical procedure. A flow path may be defined between the input aperture and the output aperture and include a sensor segment. At least two flexible membranes may be coupled to the sensor segment and in fluid communication with the flow path.

These and other features, objects and advantages of the present disclosure will become apparent upon reading the following description thereof together with reference to the accompanying drawings.

### BRIEF DESCRIPTION OF THE DRAWINGS

FIG. 1 is a top perspective view of a pump cassette in accordance with an aspect of the present disclosure;

FIG. 2A is a first side cross-sectional view of a pump cassette showing an exemplary flow path in accordance with an aspect of the present disclosure;

FIG. 2B is a second side cross-sectional view of a pump cassette showing a flow path in accordance with an aspect of the present disclosure;

FIG. 2C is a cross-sectional perspective view of a sensor segment of a pump cassette in accordance with an aspect of the present disclosure;

FIG. 2D is a front cross-sectional view of a sensor segment of a flow path for a pump cassette in accordance with an aspect of the present disclosure;

FIG. 2E is a rear cross-sectional view of a sensor cap at least partially defining a sensor segment of a flow path for a pump cassette in accordance with an aspect of the present disclosure;

FIG. 2F is a front perspective view of a flexible membrane in accordance with an aspect of the present disclosure;

FIG. 3A is a second side cross-sectional view of a pump cassette showing an exemplary flow path in accordance with an aspect of the present disclosure;

FIG. 3B is a second side cross-sectional view of a pump cassette showing an exemplary flow path in accordance with an aspect of the present disclosure;

FIG. 3C is a second side cross-sectional view of a pump cassette showing an exemplary flow path in accordance with an aspect of the present disclosure;

FIG. 3D is a second side cross-sectional view of a pump cassette showing an exemplary flow path in accordance with an aspect of the present disclosure;

FIG. 3E is a second side cross-sectional view of a pump cassette showing an exemplary flow path in accordance with an aspect of the present disclosure;

FIG. 4 is a partially schematic view of a surgical pump system that includes a surgical pump assembly, a pump cassette, and a control system in accordance with an aspect of the present disclosure; and

FIG. 5 is a control system for a surgical pump system in accordance with an aspect of the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

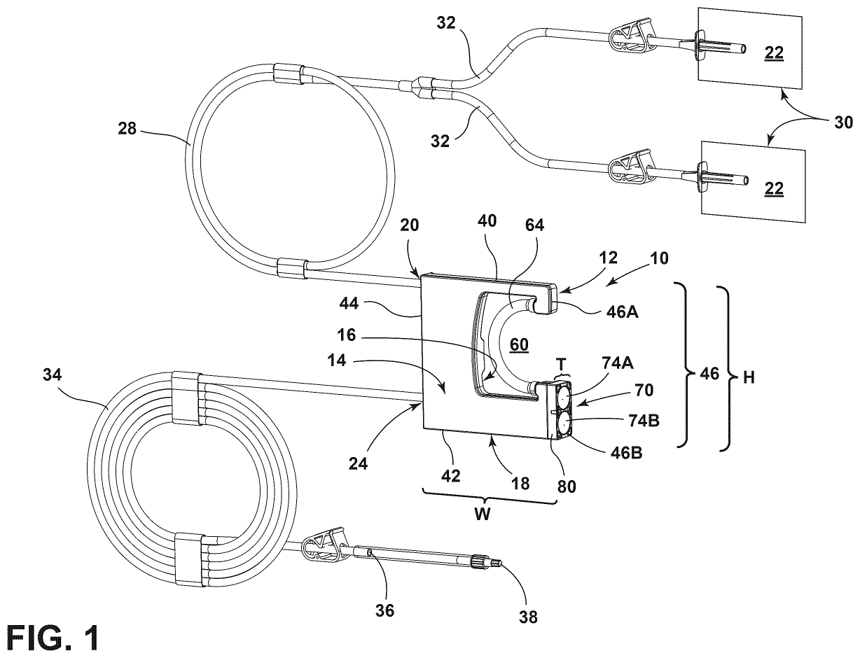

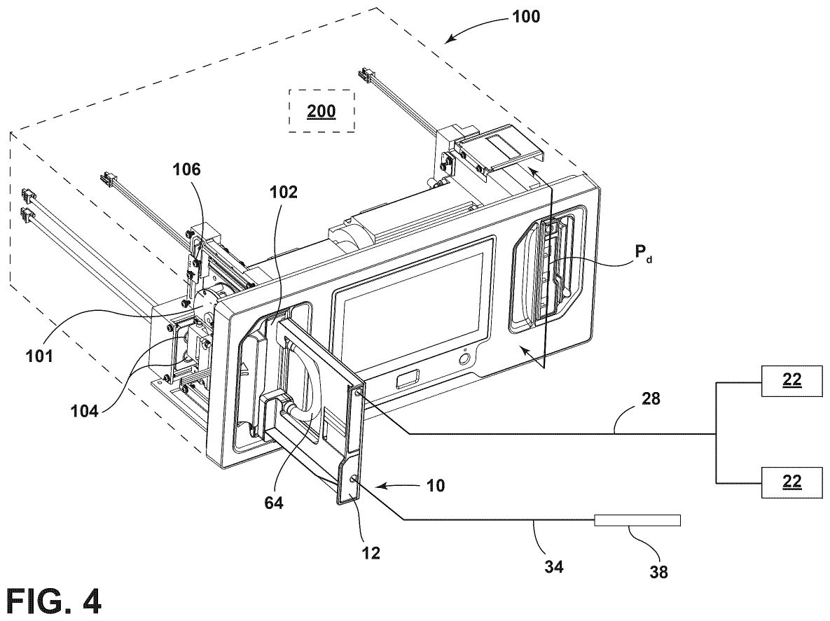

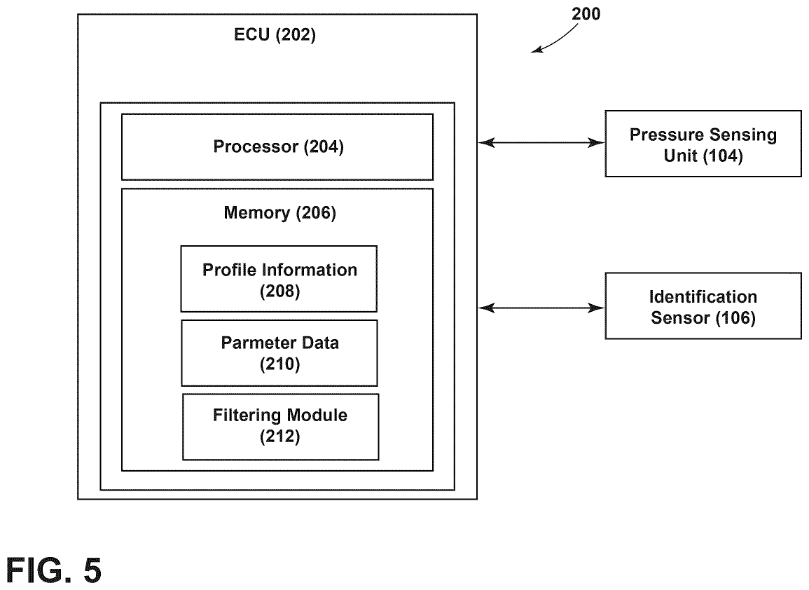

Referring generally to FIGS. 1-5, the disclosure generally provides for a variety of features and operating methods related to a surgical pump system that may include one or more of a pump cassette 10 (FIGS. 1-4), a surgical pump assembly 100 (FIG. 4), and a control system 200 (FIG. 5) for the surgical pump system. As demonstrated in reference to the following exemplary devices and assemblies, the surgical pump assembly 100 may supply fluid with limited variations in pressure or pulses that may be contrary to the operation of the surgical pump 100 in various use cases. Limiting the pulsation may further enable improved pressure control of the fluid supplied in various surgical procedures, such as arthroscopic procedures, which may support improved patient outcomes. Accordingly, the devices, systems, and methods described in the following disclosure may be implemented to provide improved operation and methods to support a variety of surgical procedures.

The surgical pump system may include various components and structures that provide flow rate sensing capabilities and the transfer of surgical solution at stabilized, constant, near constant, or controlled flow rates during a surgical procedure. By accurately controlling the flow rate, the system may further control the pressure of the fluid delivered to a distended cavity associated with a surgical site. Accordingly, the pump cassette 10 and/or surgical pump assembly 100 may include one or more sensors that are readable by the control system 200 to provide up-to-date feedback on the fluid pressure within the pump cassette 10, which may be representative of the flow rate and the pressure in the patient cavity. In this manner, the surgical pump system may be utilized to monitor (e.g., sample, detect, and/or otherwise obtain) and ensure that a desired flow rate profile and pressure may be maintained throughout the course of a surgical procedure.

As demonstrated in various examples, the surgical pump assembly 100 may be configured as a peristaltic pump with rollers that engage a tubing component of the pump cassette 10. While peristaltic pumps may be convenient for sterile applications, their operation may be associated with variations in pressure, which may result in turbulence and intermittent disruptions in the fluid flow. For example, as the rollers squeeze the tubing component to draw fluid through the pump cassette 10 and to the patient without interacting with any fluids supplied or returned from the patient. However, the interaction between the rollers and tubing component can cause pulsations and/or otherwise nonconstant or non-stabilized flow. In some implementations, the pump cassette 10 may include a tortuous flow path that may include one or more of turns, branches, and air pockets that abate, absorb, and/or dampen any pulsations and/or inconsistencies of the flow rate. The flow rate may be defined as the total amount of surgical solution 22 output or input over a given time frame. The pulsations of flow may be defined as patterns or the frequency of changing flow rates, flow amplitudes, and/or the like.

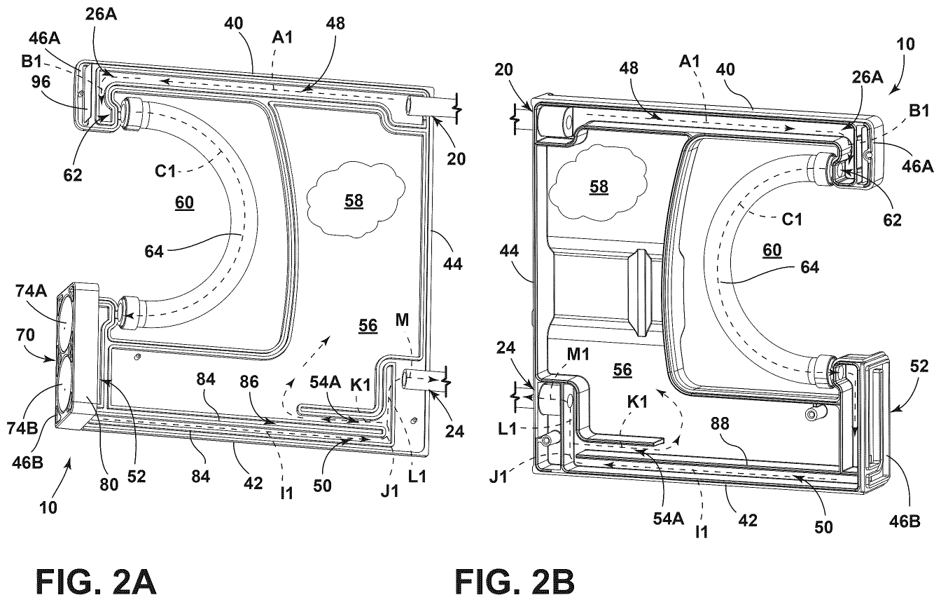

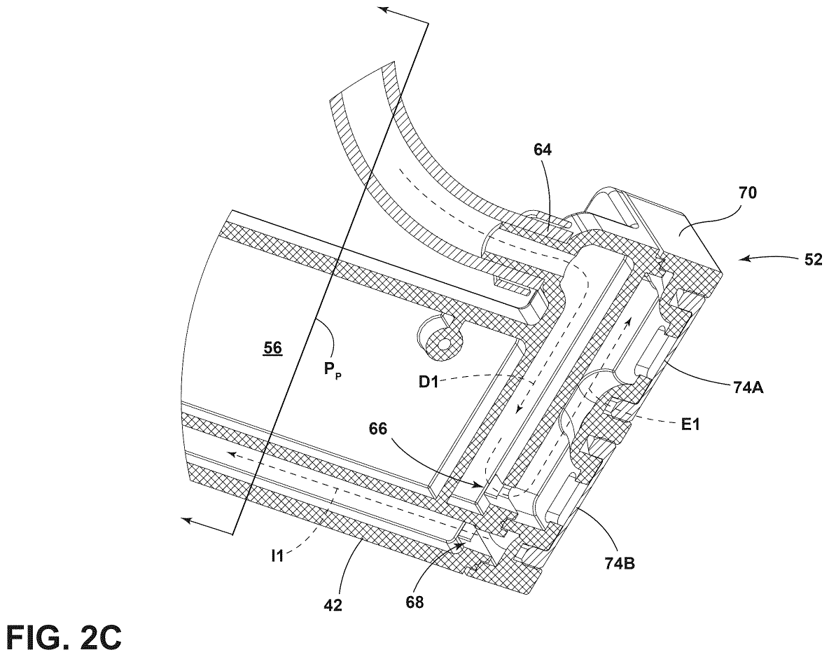

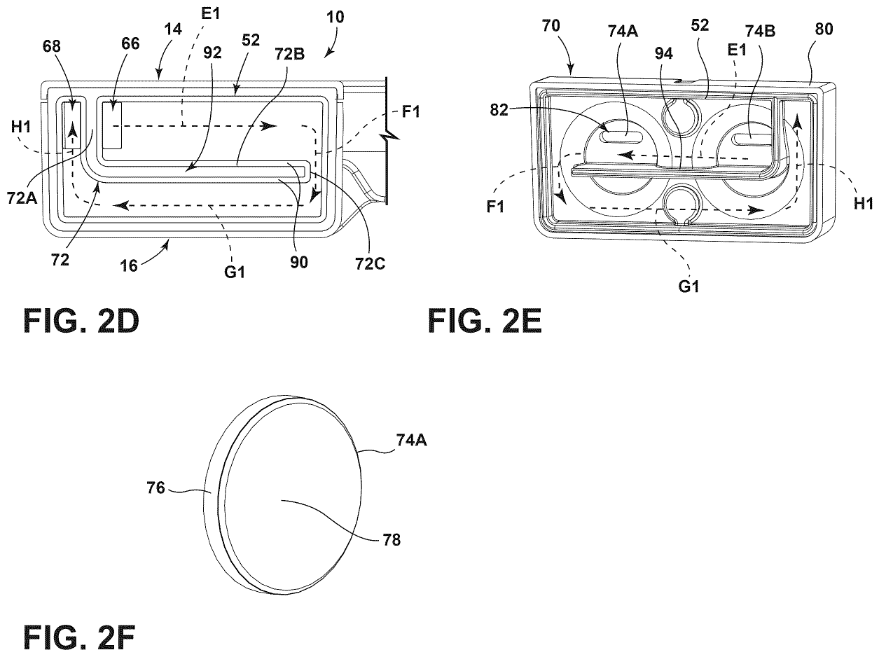

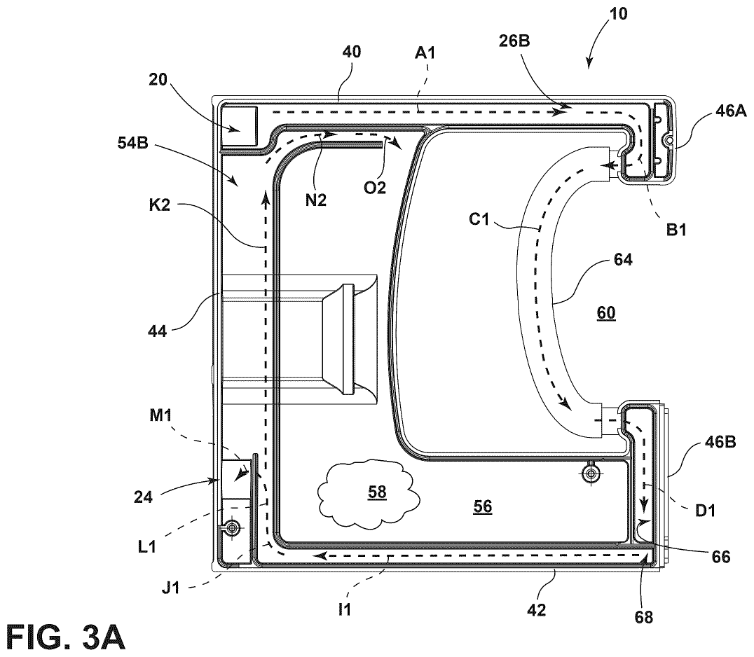

With reference now to FIGS. 1-2B, the pump cassette 10 may include a cassette housing 12 including a first major wall 14 and a second major wall 16 spaced from the first major wall 14 by a sidewall 18. The pump cassette 10 may be implemented for controlling a fluid supply to a patient cavity to maintain a stabilized, constant, or near constant flow rate. In the example shown, surgical solution 22 may be supplied via an input aperture 20 (e.g., a first tube connection) may be defined by the cassette housing 12 and configured to receive the surgical solution 22 during a medical procedure. An output aperture 24 (e.g., a second tube connection) may be defined by the cassette housing 12 and configured to expel the surgical solution 22 during the medical procedure. A flow path 26A-26E may be defined between the input aperture 20 and the output aperture 24. In operation, the flow path 26A-26E may include one or more structures, components, and/or the like that provide flow rate sensing capabilities and the transfer of surgical solution at stabilized flow rates. An input tube 28 may be coupled to input aperture 20 (e.g., via a collar, adapter, or connection member) and extend to a surgical solution reservoir 30 (e.g., a saline bag). In some implementations, the input tube 28 may extend from the input aperture 20 to and/or be selectively coupled to a branched end with two or more tube branches 32 that each connect to a different surgical solution reservoir 30 to increase the supply of available surgical solution 22 over the course of a surgical procedure.

An output tube 34 may be coupled to (e.g., via a collar, adapter, or connection member) and extend from the output aperture 24 to a fluid port end 36. The fluid port end 36 may include and/or be selectively coupled to a fluid distribution tool 38. The fluid distribution tool 38 may be configured to distribute the surgical solution 22 to the patient during the medical procedure. In some implementations, the fluid distribution tool 38 may include a cannula. In some implementations, the fluid distribution tool 38 may be configured for other functionalities which may be in addition to the fluid distribution. For example, the fluid distribution tool 38 may include a nano-scope and/or other surgical tools. In operation, surgical solution 22 may be transferred from the one or more surgical solution reservoirs 30, through the input tube 28, the pump cassette housing 12, and the output tube 34 and to the patient. The surgical solution 22 may be supplied to the patient to distend a joint space or other cavities formed in the body to provide an internal space in which a surgical procedure may be performed (e.g., endoscopically, arthroscopically, etc.).

In various minimally invasive surgical procedures, patient outcomes may be improved by accurately controlling the fluid pressure of the surgical fluid supplied to the patient cavity. For example, in cases where the fluid pressure is not accurately controlled, variations in fluid pressure may result in tissue damage, excessive bleeding, joint capsule injury, and/or fluid extravasation, which may increase recovery time and adversely impact patient outcomes. Accordingly, accurately controlling the flow rate and corresponding flow pressure of the surgical fluid supplied to the patient may be beneficial in ensuring optimal conditions to successfully perform a surgical procedure while minimizing variations in pressure. As provided in the following disclosure, the surgical cassette provides several features that may be implemented independently or in various combinations to improve the operation of the pump and limit or accurately control variations in pressure and flow rate of the surgical fluid.

In various implementations, the features of the pump cassette 10 may be implemented to limit or control the fluctuations in pressure and flow rate may be integrated in housing 12. Accordingly, the housing 12 is first described in detail for reference. The sidewall 18 of the pump cassette housing 12 may include a first lateral sidewall section 40 and a second lateral sidewall section 42 spaced by a first longitudinal sidewall section 44 and a second longitudinal sidewall section 46. In some implementations, the input aperture 20 and the output aperture 24 may be defined by the sidewall 18, and, more particularly, the first longitudinal section 44. The pump cassette housing 12 may be generally rectilinearly shaped, where two, three, or each of the first lateral sidewall section 40, the second lateral sidewall section 42, the first longitudinal sidewall section 44, and/or the second longitudinal sidewall section 46 may generally extend linearly. In some implementations, the first lateral sidewall section 40 and the second lateral sidewall section 42 may, at least in part, be planar and/or parallel. The first longitudinal sidewall section 44 may be perpendicular to the first lateral sidewall section 40 and the second lateral sidewall section 42. The first longitudinal sidewall section 44 may also, at least in part, be planar.

Without limiting the disclosure to a particular operational orientation, the pump cassette housing 12 may include a thickness “T” defined by the sidewall 18, a width “W” defined between the first longitudinal sidewall section 44 and the second longitudinal sidewall section 46, and a height “H” defined between the first lateral sidewall section 40 and the second lateral sidewall section 42. In some implementations, thickness T may be substantially less (e.g., by a ratio of 1:4 or less, 1:6 or less, 1:8 or less) of the width W and/or height H. In this manner, the cassette housing 12 can be conveniently stored, easily held, and manipulated for coupling and decoupling from the pump assembly 100.

With reference now to FIGS. 2A and 2B, in some implementations, the flow path 26A-26E may be defined within the pump cassette housing 12. As will be described in detail, the flow path 26A-26E may be tortuous to abate or dampen pulsations of the flow rate which may appear as a result of the engagement between the rollers of a peristaltic pump and the tubing component. The flow path 26A-26E may provide many segments and/or routing sections with turns and shapes that generally change the direction of momentum of the surgical solution 22 and mitigate flow rate pulsations. Further, the segments and/or routing sections may include one or more pulsation branches and air pockets that dampen pulsations of the surgical solution 22 during operation. The flow path 26A-26E may include an input flow path segment 48 extending from the input aperture 20 and may further extend at least partially along (e.g., parallel to and/or partially defined by) the first lateral sidewall section 40 to one of the opposing sections of the second longitudinal sidewall section 46. The flow path 26A-26E may further include an output flow path segment 50 extending at least partially along the second lateral sidewall section 42 (e.g., parallel to and/or partially defined by) to the output aperture 24. A sensor segment 52 may be disposed between the input flow path segment 48 and the output flow path segment 50. In this manner, surgical solution 22 may be introduced into the input flow path segment 48, flow through the sensor segment 52 to the output flow path segment 50, and through the output flow path segment 50 toward the patient.

To abate or dampen flow rate pulsations, turbulence, and prevent air bubbles that can negatively impact surgeon vision and, in some implementations, achieve near laminar flow of the surgical solution 22, the flow path 26A-26E may be tortuous and include several different aspects that limit variations in flow rate and pressure pulsations that may be attributed to turbulent flow. For example, the flow path 26A-26E may include one or more pulsation abatement flow path branches between the input aperture 20 and the output aperture 24 for pulse abatement of the surgical solution 22. For example, one or more pulse abatement flow path branches may be located along the input flow path segment 48, the output flow path segment 50, and/or the sensor segment 52. The one or more pulse abatement flow path branches may extend from a portion of the primary flow path 26A-26E transversely (e.g., perpendicularly, at an acute angle, at an obtuse angle, or in an opposite direction). In this manner, the surgical solution 22 entering the one or more pulse abatement flow paths may be traveling in a different direction than the surgical solution 22 passing through a proximate portion of the primary flow path 26A-26E. In some implementations, the one or more pulsation abatement flow path branches may include a pulsation abatement flow path branch 54A proximate the output aperture 24. More particularly, the pulsation abatement flow path branch 54A may extend from the output flow path segment 50 to a cavity 56. The cavity 56 may be disposed between inner edges or walls of the input flow path segment 48, the output flow path segment 50, and the sensor segment 52. In some implementations, the pulsation abatement flow paths described herein may be the only source of fluid communication between the flow path 26A-26E and the cavity 56. In this way, the pulsation abatement flow paths described herein may be described as extending from and/or bridging fluid communication between the flow path 26A-26E to the cavity 56. In some implementations, an air pocket 58 may be located in the cavity 56. For example, the air pocket 58 may include pressurized gas or air in fluid communication with the flow path 26A-26E that may dampen and/or absorb pulsations of fluctuating pressure of the surgical solution 22 to obtain stabilized, constant, or near constant flow rates with limited to no bubble formation. In this manner, the air pocket 58 may be hermetically sealed within the cavity 56 and pressurized. In some implementations, the quantity of air/medium in the air pocket 58 stays the same but the volume changes (e.g., compression and expansion) as a result of changes in pressure internal to the pump cassette 10. In this manner, the fluctuating volume of the air pocket 58 may function like a damper to absorb pulsations of fluctuating pressure of the surgical solution 22. The pulsation abatement flow path branch 54A in the flow path 26A of an exemplary arrangement depicted in FIGS. 2A-2E may extend at least partially along (e.g., internally from) the second lateral sidewall section 42 in a direction that may be different (e.g., transverse and/or opposite) the output flow path segment 50. In this manner, surgical solution 22 and/or pressure imparted by the surgical solution 22 entering the cavity 56 may be traveling in a different direction than the surgical solution 22 passing through the output flow path segment 50.

With continued reference to FIGS. 2A and 2B, details of exemplary surfaces and structures that may be implemented in the pump cassette 10 are provided. The second longitudinal sidewall section 46 may include a pair of opposing sections 46A, 46B extending to a bay 60 that extends towards the first longitudinal sidewall section 44. An intermediary flow path segment 62 may extend from the input flow path segment 48 to the output flow path segment 50. The intermediary flow path segment 62 may include a bridge tube 64 that may be coupled to the opposing sections 46A, 46B of the second longitudinal sidewall section 46 and may be bowed towards the first longitudinal sidewall section 44 within the bay 60. While not explicitly limited thereto, the pump assembly 100 may be configured as a peristaltic pump. In this manner, the bridge tube 64 may be formed of a flexible material and configured to be engaged by the peristaltic pump upon coupling the pump cassette 10 to the pump assembly 100. However, it is contemplated that the pump assembly 100 and pump cassette 10 may both be configured to pressurize and transfer surgical solution 22 by principles other than the peristaltic rollers. The bay 60 may provide space for the peristatic pump, for example, a roller assembly or rollers 101 (FIG. 4) that may actively engage the bridge tube 64 upon substantially linear insertion into the pump assembly 100. The rollers 101 may be configured to squeeze the bridge tube 64 to draw the surgical solution 22 through the pump cassette 10 and to the patient without interacting with or exposing the surgical solution 22 to any mechanical parts or opportunities for contamination. However, because the interaction between the rollers and tubing component may cause pulsations, the flow path 26A-26E, routing sections, and the air pocket 58 can be implemented to mitigate, dampen, absorb, and/or abate the pulsations for a substantially constant or stabilized flow rate.

With reference now to FIGS. 2C-2E, in addition to pulsation mitigation, the surgical pump system may be configured to monitor (e.g., sample, detect, and/or otherwise obtain) the flow of the surgical solution 22 to maintain a desired flow rate over the course of the surgical procedure. The sensor segment 52 may be utilized for measuring the flow rate and, more particularly, the pressure within the flow path 26A-26E. The sensor segment 52 may include a sensor segment input opening 66, a sensor segment output opening 68, and a pressure module 70 configured to react to changes in pressure. A sensor segment wall 72 may define a portion of the flow path 26A-26E between the sensor segment input opening 66 and the sensor segment output opening 68. The sensor segment wall 72 may include a first sensor segment wall portion 72A located between the sensor segment input opening 66 and the sensor segment output opening 68. In some implementations, the sensor segment input opening 66 and the sensor segment output opening 68 may abut and/or be in close proximity (e.g., within 3 mm or less, within 2 mm or less, within 1 mm or less) opposite sides of the first sensor segment wall portion 72A The sensor segment wall 72 may further include a second sensor segment wall portion 72B extending transversely (e.g., perpendicularly) from the first sensor segment wall portion 72A to a sensor segment wall terminal end 72C. The flow path 26A-26E follows the sensor segment wall 72 from the sensor segment input opening 66 past the sensor segment wall terminal end 72C and to the sensor segment output opening 68. The pressure module 70 may be located along the second longitudinal sidewall section 46 and, more particularly, one of the opposing sections 46A, 46B. In some implementations, the second longitudinal sidewall section 46 may be a front side of the pump cassette housing 12 that faces the pump assembly 100 during coupling. In this manner, the pressure module 70 may face the pump assembly 100 during insertion (e.g., proximate the bridge tube 64).

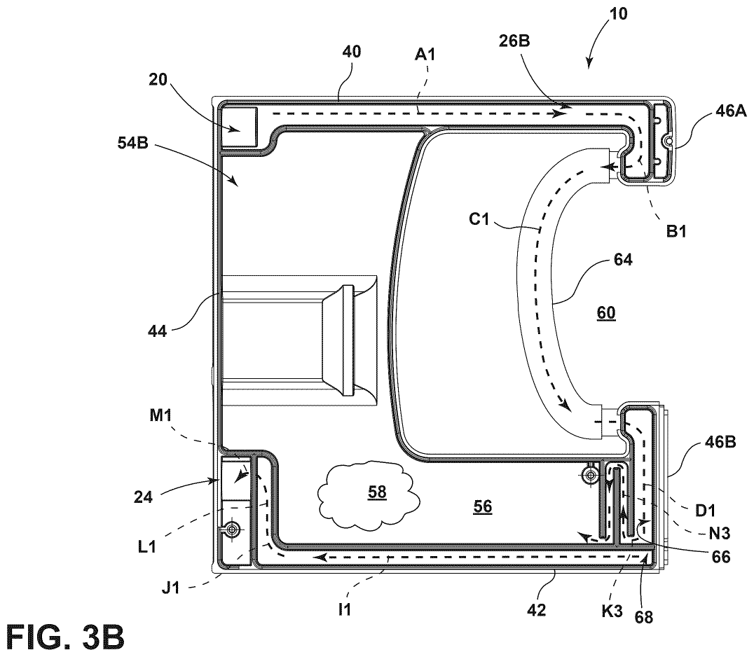

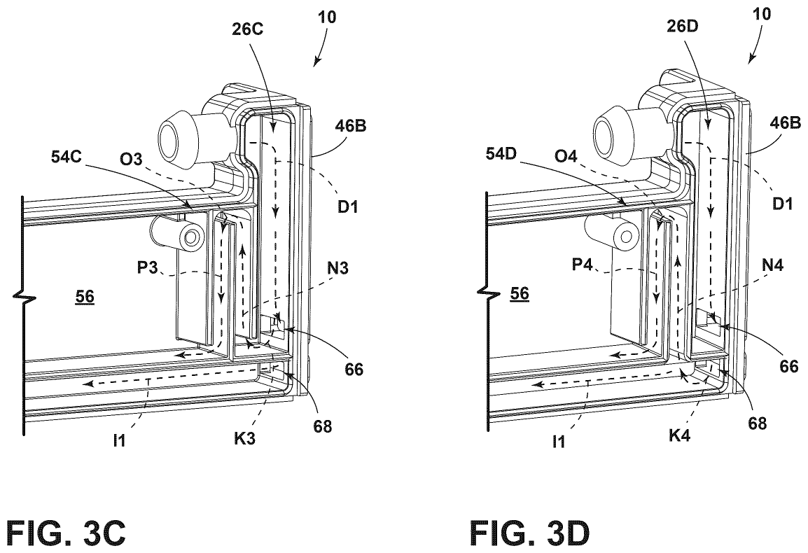

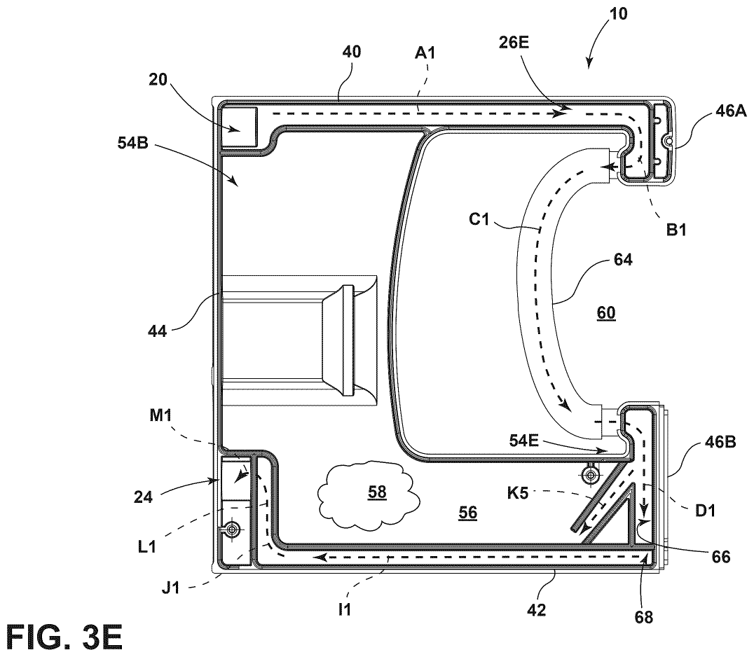

With reference now to FIGS. 2A-3G, the exemplary flow path 26A-26E may include a variety of configurations each having different arrangements of a series of route sections between the input aperture 20 and the output aperture 24. Each of the route sections may serve to communicate the surgical solution 22 through the pump cassette 10 from the inlet (e.g., input aperture 20) to the outlet (e.g., output aperture 24) via the bridge tube 64. As these features are described, the corresponding function of each segment is also discussed for clarity. Unless otherwise explicitly stated, these various configurations of the flow paths 26A-26E may be implemented with all the features, elements, and components as described in reference to the pump cassette 10. Likewise, unless otherwise explicitly stated, these various configurations of the flow paths 26A-26E may be utilized with and configured to communicate with the pump assembly 100 and control system 200. Further, other flow paths may include combinations of features, paths, and relative shapes from different ones of the various configurations of the flow paths 26A-26E without departing from the scope of the subject disclosure. Generally, it should be understood that the various flow path configurations 26A-26E may mitigate, dampen, absorb, prevent the formation of air bubbles (e.g., cavitations), and/or abate the pulsations for a substantially constant or stabilized flow rate by one, two, or more substantial changes in the direction of momentum of the surgical solution 22. With initial reference now FIGS. 2A-2E, a flow path 26A of an exemplary arrangement is depicted.

Starting at the input aperture 20 (FIGS. 2A and 2B), surgical solution 22 may be introduced into the pump cassette housing 12 via the input tube 28. Within the cassette housing 12, a first route section A1, which may be associated with the input flow path segment 48, may carry the surgical solution 22 in a linear or substantially linear path from the first longitudinal sidewall section 44 to the second longitudinal sidewall 46 (e.g., the first opposing section 46A). At the first opposing section 46A, the flow path 26A may define a second route section B1 that may be transverse (e.g., substantially perpendicular) to the first route section A1 and may pass to the bridge tube 64, which may define a third route section C1. In the third route section C1, the surgical solution 22 may be squeezed by rollers 101 of the peristaltic pump and urged forward. The third route section C1 may terminate at the second opposing section 46B where the surgical solution 22 may flow through the sensor segment 52. As discussed herein, the terms first, second, third, etc., may be used to clearly refer to various features and/or operations. These enumerated terms are not necessarily indicative of priority, order, frequency, or count of the corresponding apparatuses or methods unless specified. Accordingly, the numbered references shall be considered similar to the reference numbers, to clearly describe the text in reference to the corresponding figures and content of the application.

As best illustrated in FIGS. 2C-2E, the sensor segment 52 may define a fourth route section D1, which may be spaced from the sensor cap 80 and travel from a bay 60 side of the sensor segment 52 towards the second lateral sidewall section 42 to the sensor segment input opening 66. The fourth route section D1 may be transverse or perpendicular to the third route section C1 and substantially change a direction of momentum of the surgical solution 22 to mitigate the pulsations. In some implementations, the pulse abatement flow path branch 54A or a different pulse abatement branch may extend from the fourth route section D1 into the cavity 56. The pulse abatement flow path branch 54A may include one or more turns relative to the fourth route section D1 that changes the momentum of the surgical solution 22 entering the cavity 56. After passing through the sensor segment input opening 66, the flow path 26A may define a fifth route section E1 that travels back (e.g., in a transverse or opposite direction from the fourth route section D1) from the second lateral sidewall section 42 towards the bay 60 along the sensor segment wall 72. At the sensor segment wall terminal end 72C, the surgical solution 22 may be communicated along a first transverse turn (e.g., perpendicularly) along a sixth route section F1 and a second transverse turn (e.g., perpendicularly) along a seventh route section G1. As shown, the fifth route section E1 may extend through an interface between the pump cassette 10 shown in FIG. 2C and the sensor cap 80 shown in FIG. 2E. In some implementations, the flow path in sections A1-D1 may be located on the same primary plane Pₚ.

As shown in FIGS. 2D and 2E, the fluid path extending through the sections E1, F1, G1, and H1 may diverge from the primary plane Pₚ associated with the preceding sections A1-D1 and the following sections H1-M1. For example, as the surgical solution 22 transitions from the fourth routing section D1 to the fifth routing section E1, the direction of the fluid flow may change transversely in a direction that would appear to protrude from the drawing at the end of the arrow shown in FIG. 2C. More particularly, the second sensor segment wall portion 72B may be located closer to at least one major wall 14, 16 (e.g., the second major wall 16) than or bottom or top surface of the flow path in preceding sections A1-D1. In this configuration, the fifth, sixth, and seventh routing sections E1-F1 may diverge from a primary flow path plane between the major walls 14, 16 of the housing 12 (e.g., along the thickness of the housing 12). The surgical solution 22 in the fifth route section E1 may extend in a first direction along the sensor segment wall 72 and the surgical solution in the seventh route section G1 may travel a second direction (e.g., opposite the first direction) along an opposite side of the sensor segment wall 72. After the surgical solution 22 passes the sensor segment wall 72 it may enter an eighth route section H1 that may extend from the seventh route section G1 (e.g., perpendicularly) to the sensor segment output opening 68. In some implementations, route sections F1 and H1 may travel in opposite directions.

As best depicted in FIGS. 2A and 2B, after passing through the seventh route section G1 and sensor segment output opening 68, shown in FIG. 2E, the path of the surgical solution 22 may be at least partially or fully offset from the primary plane Pₚ and then return back to the primary plane Pₚ through the eighth route section H1 and may further travel to a ninth route section I1 as shown in FIG. 2C. In the ninth route section I1, the surgical solution 22 may flow along the output flow path segment 50 (e.g., linearly and along the primary plane Pₚ) before reaching the pulsation abatement flow path branch 54A. In some implementations, the pulse abatement flow path branch 54A or a different pulse abatement branch may extend from the ninth route section I1 into the cavity 56. The pulse abatement flow path branch 54A may include one or more turns relative to the ninth route section I1 that changes the momentum of the surgical solution 22 and/or pressure imparted by the surgical solution 22 entering the cavity. More particularly, at the end of the ninth route section I1, the flow path may include a tenth route section J1, which may be arranged transverse (e.g., perpendicular) to the ninth route section I1. At the tenth route section J1, the surgical solution 22 may travel to either or be split between an eleventh route section K1 or a twelfth route section L1. The surgical solution 22 in the eleventh route section K1 may travel in an opposite direction to the surgical solution 22 in the ninth route section I1, where the surgical solution 22 and/or pressure imparted by the surgical solution 22 may be guided by the pulsation abatement flow path branch 54A into the cavity 56. In some implementations, the pump cassette 10 may be configured to be inserted into the surgical pump assembly 100 (e.g., the cassette port 102) with the sidewall section 42 facing downwardly. In such implementations, surgical solution 22 entering the eleventh route section K1 must overcome the forces of gravity. As a result, the abatement flow path branch 54A and/or any route sections associated therewith may function as or otherwise be referred to as a pressure head.

In addition to the various route sections, flow path branches, and different tortuous flow paths, the flow path may provide additional features for pulsation abatement and/or flow rate stabilization. For example, during operation, the difference in the height of the pressure head may allow the periodic excess energy causing the pulses to be absorbed in the vertical section. The pressure head may therefore work in conjunction with the variable pressurization of air in the chamber 56 to mitigate the pulses of the resulting fluid flow. The cavity may form an air pocket 58 or pillow in which remaining pulsations or pressure fluctuations may further be dampened by the air pocket 58. In the twelfth route section L1, the surgical solution 22 may continue to flow along the flow path at a transverse angle (e.g., perpendicularly) from the ninth route section I1. The twelfth route section L1 may continue into a thirteenth route section M1. The thirteenth route section M1 may be transverse (e.g., perpendicular) to the twelfth route section L1. The thirteenth route section M1 may lead the surgical solution to the output aperture 24 where the surgical solution 22 may continue to travel along the output tube 34 to the surgical site. In some implementations, the cassette housing 12 may be configured to be inserted into the pump assembly 100 in an orientation where the first lateral sidewall section may be facing upwards. In this manner, it should be appreciated that the flow of surgical solution 22 through the cassette housing 12 may be assisted by gravity. Further, different pulsation abatement sections (e.g., sensor segment 52) of the flow path 26A may utilize gravity to impart a force of the surgical solution 22 and reduce flow turbulence. In addition, the pulse abatement flow path branch 54A or a different pulse abatement branch may extend from any of the above-described route sections A1-M1 into the cavity 56.

With reference now to FIGS. 2D-2F, to ensure the pressure module 70 is operating effectively and accurately, the pressure module 70 may include two detection modules that may be individually readable and independently report the pressure of the fluid in the pressure module 70 (e.g., two, three, four, or more individually readable modules). In this manner, the individual readings can be compared, averaged, and/or selectively discarded for improve accuracy and provide redundancy. For example, the pressure module 70 may include at least one flexible membrane 74A, 74B in fluid communication with the flow path 26A-26E proximate the sensor segment 52. The at least one flexible membrane 74A, 74B may be configured to expand upon increased pressure within the flow path 26A-26E and/or otherwise respond to changes in pressure. More particularly, during typical operation (e.g., conforming to the performance and operating specification of the pump assembly 100), the flexible membranes 74A, 74B may respond demonstrating similar variations in pressure that may form patterns associated with the engagement of the rollers 101 with the bridge tube 64. For example, in operation, sensors of the pump assembly 100 in connection with the flexible membranes 74A, 74B may monitor (e.g., sample, detect, and/or otherwise obtain) a common fluid chamber associated with the fifth route section E1. Accordingly, the change in pressure measured by the sensors may provide redundant or similar measurements in cases where the sensors are accurately measuring the translation, movement, flex, expansion, deflection, and/or deformation of the flexible membranes 74A, 74B. It should be appreciated that the pulse abatement flow path branch 54A or a different pulse abatement branch may be located upstream or downstream from the pressure module 70 (e.g., the sensor segment 52).

In operation, the control system 200 may monitor (e.g., sample, detect, and/or otherwise obtain) and compare the pressure measurements reported by the sensors of the pump assembly 100 to ensure the readings correlate. In cases where the pressure measurements attributed to translation of or force applied to the flexible membranes 74A, 74B correlate, the system 200 may verify that the sensors are accurately detecting the fluid pressure. However, if the pressure associated with the flexible membranes 74A, 74B do not correlate or conform in trends and patterns over time, the control system 200 may expediently identify that one or more of the sensors associated with the flexible membranes 74A, 74B is operating in error. In this way, the control system may verify that non-conforming pressure readings or pressures reported to be outside a target range for a surgical procedure are not likely attributed to sensor errors or failures.

As generally discussed previously, the at least one flexible membrane 74A, 74B may include a first flexible membrane 74A and a second flexible membrane 74B. In some implementations, the at least one flexible membrane 74A, 74B may include more than two (e.g., three, four, or more flexible membranes). In this manner, the control system 200 may monitor (e.g., sample, detect, and/or otherwise obtain) pressure readings associated with the expansion, deformation, or movement for each (e.g., both) the first flexible membrane 74A and the second flexible membrane 74B, on an individual basis (e.g., simultaneously), to ensure accuracy via averaging, filtering out inaccurate results as a result of sensing errors and/or detecting reaction irregularities of the flexible membranes 74A, 74B, translation irregularities between sensors in the pump assembly 100, and/or the like. The utilization of the two or more sensors and the corresponding flexible membranes 74A may be supported by the associated path of the tortuous path through the pump cassette 10, which may increase the lateral surface area on the insertion end along fifth, sixth, and seventh routing sections E1-F1 (e.g., the second longitudinal sidewall section 46) of the pump cassette 10. In this manner, the redundant sensors and membranes may be directed toward and compressed against sensors within the pump assembly 100 when loaded.

The at least one flexible membrane 74A, 74B may be an integrally formed body of an elastic material, such as a flexible rubber material, a PVC rubber material, a silicone material, and/or the like. In some implementations, the at least one flexible membrane 74A, 74B (e.g., the first flexible membrane 74A and the second flexible membrane 74B) may have a circular perimeter defined by a gasket rim 76 and a face portion 78 that may be thinner than the gasket rim 76. In some implementations, the face portion 78 of the at least one flexible membrane 74A, 74B (e.g., the first flexible membrane 74A and the second flexible membrane 74B) may be less than 700 μm thick. For example, the thickness of the face portion 78 may be less than 650 μm thick, less than 600 μm thick, less than 550 μm thick, less than 540 μm thick, about 533 μm thick, or between about 550 μm and 520 μm thick, including ranges between each of the above-described values. The thickness of the flexible membranes 74A, 74B can be selected based on obtaining optimal ranges of expansion and contraction for a range of achievable flow rates provided by the surgical pump system.

The sensor segment 52 may be bounded by the sidewall 18 and a sensor cap coupled to the sidewall 18. The at least one flexible membrane 74A, 74B may be coupled to the sensor cap 80 opposite the sensor segment 52 of the flow path 26A-26E. When two or more membranes 74A, 74B are utilized, they may be proximately located in a common chamber and exposed to pressure within the common chamber, such that pressure readings are expected to be equal between the flexible membranes 74A, 74B. In some implementations, the flexible membranes 74A, 74B may further extend along a common plane. The gasket rim 76 of the at least one flexible membrane 74A, 74B may be coupled to the sensor cap 80 (e.g., channels, adhesives, and/or the like). The sensor cap 80 may include at least one cap opening 82 aligned with the at least one flexible membrane 74A, 74B. The cap openings 82 may be provided such that the at least one flexible membrane 74A, 74B may be exposed to the surgical solution 22 and, more particularly, any pressure within the pump cassette housing 12. In some implementations, the number of cap openings 82 may be equal to the number of flexible membranes 74A, 74B. In some implementations, the cap openings 82 may be aligned (e.g., centrally) with the same respective regions of the flexible membranes 74A, 74B for uniformity. The cap openings 82 may each have an oblong shape. The at least one cap opening 82 may be located in the flow path 26A-26E proximate the second sensor segment wall portion 72B (e.g., along the fifth route section E1) before the sensor segment wall terminal end 72C. At least some of the surgical solution 22 that interfaces with the flexible membranes 74A, 74B may generally be retained at the impasse between the cap openings 82 and the flexible membranes 74A, 74B.

Depending on manufacturing requirements, the pump cassette housing 12 may be an integrally formed component or a plurality of separately formed components coupled to one another. For example, in the depicted arrangement in FIGS. 2A-2E, the first major wall 14 may be coupled to the second major wall 16. At least one of the first major wall 14 and the second major wall 16 may be integrally formed with the sidewall 18. In the depicted arrangement, the second major wall 16 may define the sidewall 18 (e.g., a majority, the entirety, or substantially the entirety of the sidewall 18). In this manner, the components can be initially shaped, aligned, and coupled together during manufacturing. The sensor cap 80, may likewise be formed separately and coupled to the sidewall 18. The coupling connection between the first major wall 14, the second major wall 16, and/or the sensor cap 80 may be hermetically sealed to retain the surgical solution 22 and prevent contamination. In some implementations, the coupling connection between the first major wall 14, the second major wall 16, and/or the sensor cap 80 may be welded or ultrasonically welded (e.g., a welded bond and/or an ultrasonically welded bond may be formed therebetween).

In various implementations, the flow path 26A-26E may be defined as an interior surface of the first major wall 14, the second major wall 16, the sidewall 18, and/or the sensor cap 80. For example, at least part of the input flow path 26A-26E and at least part of the output flow path 26A-26E may be defined by a rib extending inwardly from the first major wall 14 and the second major wall 16. More particularly, the first major wall 14 or the second major sidewall 16 may include a pair of parallel first ribs 84 defining a channel 86 therebetween and the other of the first major sidewall 14 and the second major wall 16 may include a second rib 88 located in the channel 86. The sensor segment 52 and sensor cap 80 may also include ribs. For example, one of the sidewall 18 and sensor cap 80 may include a pair of first sensor segment ribs 90 that may define a sensor segment channel 92 and the other of the sidewall 18 and sensor cap 80 may include a second sensor segment rib 94 disposed within the sensor segment channel 92. The ribs 88, 94 may sit within channels 86, 92 and mechanically interlock and may be sealed to form the flow path 26A-26E. Further, the opposing ribs 84, 88 and 90, 94 may add structural integrity to the pump cassette housing 12 from external and/or internal forces. These interlocking and opposing components may be utilized to facilitate a robust and sturdy design that maintains a sealed flow path 26A-26E during the operational life of the pump cassette 10 while keeping manufacturing simple and efficient.

The surgical system may utilize different types of pump cassettes with different optimized flow requirements, different membrane 74A, 74B dimensions, numbers, or materials, and other differences that can impact optimal usage. In this manner, it may be beneficial for the pump assembly 100 to identify and classify which of the different types of pump cassettes are loaded into the pump assembly 100 to recommend or automatically implement pump settings. For example, an identifier module 96 (FIG. 2A) may be located in the pump cassette housing 12. The identifier module 96 may include identification data associated with, for example, information related to the pressure sensing module 70 (e.g., the number and type of modules). The identifier module 96 may further include information related to the flow path 26A-26E, the air pocket 58, the size, shape, and other information related to the pump cassette housing 12. As explained previously, the pressure module 70 may be located along one of the opposing sections 46A, 46B. In some implementations, the identifier module 96 may be located in the other one of the opposing sections 46A, 46B from the pressure module 70. The identifier module 96 may include any readable data that may be detectable by the pump assembly 100 or a component associated therewith and translated by the control system 200. In some implementations, the identifier module 96 may include a radio-frequency identification (“RFID”) tag. The control system 200 may utilize the identifier module 96 to detect and identify characteristics of the pump cassette 10 and recommend and/or automatically implement various pump settings (e.g., flow rate or pressure) or sensor settings (e.g., membrane translation profiles).

With reference now to FIGS. 3A-3G exemplary flow paths 26B-26E having different route sections are depicted. It should be appreciated that, unless otherwise explicitly illustrated, these flow paths 26B-26E may be utilized with any of the features, functions, and elements described in reference to the pump cassette 10 throughout the figures. For purposes of clarity, unless explicitly stated, undescribed portions or route sections of the flow paths 26B-26E may be the same as those described in reference to the flow path 26A. In addition, like route sections throughout the exemplary flow paths 26A-26E and components of the pump cassette 10 described herein are designated with the same reference numerals.

With reference now particularly to FIG. 3A, another example of a flow path 26B is depicted. The flow path 26B may include all of the above-described route sections A1-M1 as described in reference to the flow path 26A depicted in FIGS. 2A-2E. However, the flow path 26B may include a pulsation abatement flow path branch 54B in a different location relative to the route sections. More particularly, the flow path 26B may include the first route section A1, which may be associated with the input flow path segment 48, and may carry the surgical solution 22 in a linear or substantially linear path from the first longitudinal sidewall section 44 to the second longitudinal sidewall 46 (e.g., the first opposing section 46A). At the first opposing section 46A, the flow path 26A may define the second route section B1 that may be transverse (e.g., substantially perpendicular) to the first route section A1 and may pass to the bridge tube 64, which may define the third route section C1. In the third route section C1, the surgical solution 22 may be squeezed by rollers 101 of the peristaltic pump and urged forward. The third route section C1 may terminate at the second opposing section 46B where the surgical solution 22 may flow through the sensor segment 52 at the fourth route section D1. While not shown in FIG. 3A, the route sections E1-H1 in the sensor segment 52 may be the same or substantially the same as those described in reference to the flow path 26A depicted in FIGS. 2A-2E.

After passing through the sensor segment 52 (e.g., route sections E1-H1), the flow path 26B may continue along the ninth route section I1 where the surgical solution 22 may flow along the output flow path segment 50 (e.g., linearly and along the primary plane Pₚ). The flow path 26B may continue to the tenth route section J1, which may be arranged transverse (e.g., perpendicular) to the ninth route section I1 before traveling to the twelfth route section L1. The twelfth route section L1 may be substantially parallel to the tenth route section J1 and terminate at a juncture that may include the thirteenth route section M1 where the surgical solution 22 exits the output aperture 24 and the eleventh route section K2. Unlike the flow path 26A depicted in FIGS. 2A-2E, the pulsation abatement flow path branch 54B and the corresponding eleventh route section K2 may be upstream from the output aperture 24. More particularly, the eleventh route section K2 may extend parallel or substantially parallel to the twelfth route section L1 and perpendicular or substantially perpendicular to the thirteenth route section M1. The eleventh route section K2 travels to a fourteenth route section N2 that may include a bend where a surgical solution may be routed transversely from the eleventh route section K2. In some implementations, the pump cassette 10 may be configured to be inserted into the surgical pump assembly 100 (e.g., the cassette port 102) with the sidewall section 42 facing downwardly. In such implementations, surgical solution 22 entering the eleventh route section K2 must overcome the forces of gravity. As a result, the abatement flow path branch 54B and/or any route sections associated therewith may function as or otherwise be referred to as a pressure head. During operation, the difference in the height of the pressure head allows the periodic excess energy causing the pulses to be absorbed in the vertical section. The pressure head works in conjunction with the variable pressurization of air in the chamber 56 to mitigate the pulses of the resulting fluid flow. In some implementations, at least part of the fourteenth route section N2 extends perpendicularly or substantially perpendicularly to the first route section A1. In some implementations, the same sidewall/rib that defines an interior boundary of the first route section A1 also defines an exterior boundary of the fourteenth route section N2. After passing through the fourteenth route section N2, flow path 26B makes another turn at the fifteenth route section O2 into the cavity 56. In some implementations, the fifteenth route section O2 is at least partially defined by an inner wall forming the bay 60. In some implementations, surgical solution 22 in the fifteenth route section O2 travels in a substantially opposite direction than the eleventh route section K2. In other implementations, the air pocket 58 prevents the surgical solution 22 from passing beyond the eleventh route section K2 and functions as a pneumatic or fluid damper for excess surgical solution 22 and/or pressure imparted by the surgical solution 22 that has not passed into the thirteenth route section M1 and the output aperture 24. The pulsation abatement flow path branch 54B may include route sections K2, N2, and O2. In this manner, the pulsation abatement flow path branch 54B may include one or more turns, switchbacks, and/or the like to mitigate, dampen, absorb, prevent the formation of air bubbles, and/or abate the pulsations for a substantially constant or stabilized flow rate by one, two, or more substantial changes in the direction of momentum of the surgical solution 22.

With reference now to FIGS. 3B and 3C, another exemplary flow path 26C is depicted. The flow path 26C may include all of the above-described route sections A1-M1 as described in reference to the previous examples. However, the flow path 26C may include a pulsation abatement flow path branch 54C in a different location relative to the route sections than previous examples. More particularly, the flow path 26C may include the first route section A1, which may be associated with the input flow path segment 48, and may carry the surgical solution 22 in a linear or substantially linear path from the first longitudinal sidewall section 44 to the second longitudinal sidewall 46 (e.g., the first opposing section 46A). At the first opposing section 46A, the flow path 26A may define the second route section B1 that may be transverse (e.g., substantially perpendicular) to the first route section A1 and may pass to the bridge tube 64, which may define the third route section C1. In the third route section C1, the surgical solution 22 may be squeezed by rollers 101 of the peristaltic pump and urged forward. The third route section C1 may terminate at the second opposing section 46B where the surgical solution 22 may flow through the sensor segment 52 at the fourth route section D1. Unlike the previously described examples, the pulsation abatement flow path branch 54C and the associated route section K3 may be located along the fourth route section D1 at a juncture downstream from the sensor segment 52 (e.g., proximate the sensor segment input opening 66). More particularly, the route section K3 and the sensor segment input opening 66 may be at least partially (e.g., substantially or fully) aligned with the sensor segment input opening 66 on opposite sides of the fourth route section D1. In this manner, surgical solution 22 and/or pressure imparted by the surgical solution 22 that travels along the fourth route section D1 splits into substantially opposite directions. While not shown in FIGS. 3B and 3C, the route sections E1-H1 in the sensor segment 52 may be the same or substantially the same as those described in reference to the flow path 26A depicted in FIGS. 2A-2E.

Unlike the flow path 26A, the pulsation abatement flow path branch 54C and corresponding eleventh route section K3 may be downstream from the sensor segment 52. The eleventh route section K3 may extend transversely (e.g., perpendicular or substantially perpendicular) to the to the fourth route section D1. In some implementations, the pump cassette 10 may be configured to be inserted into the surgical pump assembly 100 (e.g., the cassette port 102) with the sidewall section 42 facing downwardly. In such implementations, surgical solution 22 and/or pressure imparted by the surgical solution 22 entering the eleventh route section K3 must overcome the forces of gravity. As a result, the abatement flow path branch 54C and/or any route sections associated therewith may function as or otherwise be referred to as a pressure head. During operation, the difference in the height of the pressure head allows the periodic excess energy causing the pulses to be absorbed in the vertical section. The pressure head works in conjunction with the variable pressurization of air in the chamber 56 to mitigate the pulses of the resulting fluid flow. The eleventh route section K3 travels to a fourteenth route section N3 transverse from the eleventh route section K3. In some implementations, the fourteenth route section N3 and the fourth route section D1 are parallel, such that sections D1, K3, and N3 form a first switchback. After the passing through the fourteenth route section N3, the flow path 26C may make another turn at the fifteenth route section O3 transverse to the fourteenth route section N3. In the depicted implementation, the fifteenth route section O3 and the eleventh route section K3 may be parallel to one another. The fifteenth route section O3 extends to a sixteenth route section P3 that extends transversely (e.g., perpendicular or substantially perpendicular) from the fifteenth route section O3. In some implementations, the sixteenth route section P3 extends parallel or substantially parallel to the fourteenth route section N3 and the fourth route section D1. In this manner, sections N3, O3, P3, and N3 may form a second switchback. The sixteenth route section P3 may extend from the fifteenth route section O3 and into the cavity 56. In this manner, the pulsation abatement flow path branch 54C may include the route sections K3 and N3-P3. The one or more turns, switchbacks, serpentine patterns, and/or the like to mitigate, dampen, absorb, prevent the formation of air bubbles, and/or abate the pulsations for a substantially constant or stabilized flow rate by one, two, or more substantial changes in the direction of momentum of the surgical solution 22.

With continued reference to FIGS. 3B and 3C, surgical solution 22 that has passed by the pulsation abatement flow path branch 54C (e.g., the route section K3) and through the sensor segment 52 (e.g., route sections E1-H1), may continue along the ninth route section I1 where the surgical solution 22 may flow along the output flow path segment 50 (e.g., linearly and along the primary plane Pₚ). The flow path 26C may continue to the tenth route section J1, which may be arranged transverse (e.g., perpendicular) to the ninth route section I1 before traveling to the twelfth route section L1 and out through the thirteenth route section M1.

With reference now to FIG. 3D, yet another exemplary flow path 26D is depicted. The flow path 26D may include all of the above-described route sections A1-M1 as described in reference to the previous examples. However, similar to the depicted example, the flow path 26D may include a pulsation abatement flow path branch 54D in a different location relative to the route sections than the other examples. More particularly, the flow path 26D may travel from route sections A1-H1 before defining the pulsation abatement flow path branch 54D. Unlike the flow path 26C, the pulsation abatement flow path branch 54D and the associated route section K4 may be located along the ninth route section I1 at a juncture upstream from the sensor segment 52 (e.g., proximate the sensor segment output opening 68). More particularly, the sensor segment output opening 68 may open into the ninth route section I1, where surgical solution 22 and/or pressure imparted by the surgical solution 22 travels until the pulsation abatement flow path branch 54D at route section K4. The route section K4 may be substantially closer to the sensor segment output opening 68 than the tenth route section J1. For example, the ninth route section I1 may travel along a length substantially along a majority of the second lateral sidewall section 42 and the route section K4 may be within about 15% or less of the length from the sensor segment output opening 68 (e.g., about 10% or less, about 5% or less). In some implementations, the pump cassette 10 may be configured to be inserted into the surgical pump assembly 100 (e.g., the cassette port 102) with the sidewall section 42 facing downwardly. In such implementations, surgical solution 22 and/or pressure imparted by the surgical solution 22 entering the route section K4 must overcome the forces of gravity. As a result, the abatement flow path branch 54D and/or any route sections associated therewith may function as or otherwise be referred to as a pressure head. During operation, the difference in the height of the pressure head allows the periodic excess energy causing the pulses to be absorbed in the vertical section. The pressure head works in conjunction with the variable pressurization of air in the chamber 56 to mitigate the pulses of the resulting fluid flow. While not shown in FIGS. 3B and 3C, the route sections E1-H1 in the sensor segment 52 may be the same or substantially the same as those described in reference to the flow path 26A.

The route section K4 may extend transversely (e.g., perpendicular or substantially perpendicular) to the ninth route section I1. The route section K4 travels to a fourteenth route section N4 arranged transverse from the eleventh route section K4. In some implementations, the fourteenth route section N4 and the fourth route section D1 are parallel, such that sections D1, K4, and N4 form a first switchback. After passing through the fourteenth route section N4, the flow path 26D may make another turn at the fifteenth route section O4 transverse to the fourteenth route section N4. In the depicted implementation, the fifteenth route section O4 and the eleventh route section K4 may be parallel to one another. The fifteenth route section O4 extends to a sixteenth route section P4 that extends transversely (e.g., perpendicular or substantially perpendicular) from the fifteenth route section O4. In some implementations, the sixteenth route section P4 extends parallel or substantially parallel to the fourteenth route section N4 and the fourth route section D1. In this manner, sections N4, O4, P4, and N4 may form a second switchback. The sixteenth route section P4 may extend from the fifteenth route section O4 and into the cavity 56. In this manner, the pulsation abatement flow path branch 54D may include the route sections K4 and N4-P4. The one or more turns, switchbacks, serpentine patterns, and/or the like to mitigate, dampen, absorb, prevent the formation of air bubbles, and/or abate the pulsations for a substantially constant or stabilized flow rate by one, two, or more substantial changes in the direction of momentum of the surgical solution 22.

With reference now to FIG. 3E, still another exemplary flow path 26E is depicted. The flow path 26E may include all of the above-described route sections A1-M1 as described in reference to the flow path 26A. However, similar to the flow path 26C, the flow path 26E may include a pulsation abatement flow path branch 54E in a different location relative to the route sections than the previous examples. More particularly, the flow path 26E may travel from route sections A1-C1 before defining the pulsation abatement flow path branch 54E from route section D1. Like the flow path 26C, the pulsation abatement flow path branch 54E and the associated route section K5 may be located at a juncture downstream from the sensor segment 52. However, unlike the flow path 26C, the pulsation abatement flow path branch 54E may also be located downstream from the sensor segment input opening 66 (e.g., not aligned). In some implementations, the pulsation abatement flow path branch 54E and associated route section K5 may be located proximate to route section C1. In this manner, the pulsation abatement flow path branch 54E and associated route section K5 may be at a substantially opposite side of route section D1 than the sensor segment input opening 66 and corresponding route sections associated with the sensor segment 52. The route section K5 may extend transversely from the fourth route section D1. In some implementations, the route section K5 may not be perpendicular or parallel to the fourth route section D1. More particularly, the route section K5 may extend at an acute angle from the fourth route section D1 and extend into the cavity 56 in a direction towards the second lateral sidewall section 42 (e.g., the ninth route section I1).

In some implementations, the route section K5 may have a cross-section that is larger proximate the fourth route section D1 and becomes smaller towards a termination of the route section K5 that enters into the cavity 56 and is opposite the fourth route section D1. The shrinking cross-section of route section K5, the transverse (e.g., acute) angle, and/or the like to mitigate, dampen, absorb, prevent the formation of air bubbles, and/or abate the pulsations for a substantially constant or stabilized flow rate by one, two, or more substantial changes in the direction of momentum of the surgical solution 22. In some implementations, the proximity of route section K5 to the fourth route section D1 and the acute angle effectively traps the surgical solution 22, causing little loss in pulsation control from the air pocket 58. While the route section K5 is depicted as being downstream from the sensor segment 52, it should be appreciated that, in some implementations, section K5 may extend transversely (e.g., at an acute angle or an obtuse angle) from the ninth route section I1.

With reference now to FIGS. 2A-3E, the various exemplary flow paths 26A-26E may each have shared and unique benefits. For example, having the pulsation abatement flow path branch 54E located downstream of the sensor segment 52 may be beneficial for smoothing out the pulsations prior to the interaction between the surgical solution 22 and the pressure sensing module 70 for a more consistent pressure reading. However, on the other hand, having the pulsation abatement flow path branch 54E located upstream of the sensor segment 52 may be beneficial for smoothing out the pulsations prior to removal and transfer of the surgical solution 22 through the output aperture 24. Likewise, the inclusion of multiple turns and switchbacks (e.g., serpentine patterns) that change the momentum of the surgical solution 22 can greatly reduce pulsations. However, designs with less switchbacks and turns, such as the exemplary flow path 26E may be cheaper to design and easier to assemble and may further include other pulsation remedial benefits, like the acute angle and changing cross-sectional size. It should be appreciated that the exemplary flow paths 26A and 26B may be utilized with the additional flow path branches 54C-54E and corresponding route sections of the other exemplary flow paths 26C-26E. Likewise, the exemplary flow paths 26C-26E may include any combination of upstream and downstream flow path branches 54C-54E as described in reference to the flow paths 26A-26E. It should also be appreciated that route sections associated with the flow path branches 54A-54E may not necessarily receive and/or otherwise transfer the surgical solution 22 to the cavity 56. More particularly, the flow path branches 54A-54E may abate pulsations by back pressure provided by the air pocket 58. For example, route sections K1-K5 may receive the surgical solution 22 along only a portion of the route downstream from the cavity 56, where the surgical solution 22 is prevented from further travel via backpressure. Generally, the greater the volume of surgical solution 22 that enters the flow path branches 54A-54E and associated route sections results in a greater backpressure. In some implementations, route sections N2 and O2 (FIG. 3A) may not receive surgical solution 22 or receive surgical solution 22 only along a portion downstream from the cavity 56. Similar principles apply to route sections O3 and P3 (FIG. 3B), and route sections O4 and P4 (FIG. 3D). In this manner, the route sections associated with the flow path branches 54A-54E may be referred to herein as “back pressure sections,” “damping sections,” and/or the like.

With reference now to FIG. 4, the surgical pump assembly 100 may be designed for convenient and efficient insertion of the pump cassette 10. The surgical pump assembly 100 may house one or more, each component of the control system 200 or otherwise be in operable communication with the control system 200. In some implementations, the surgical pump assembly 100 may be configured as a peristaltic pump that may use rotating bearing elements (e.g., rollers 101) to squeeze the bridge tube 64 and effectuate the flow rate of the surgical solution. In this manner, the surgical pump assembly 100 may include a cassette port 102 configured to selectively receive the pump cassette housing 12. The cassette port 102 may extend vertically such that the major walls may need to be oriented in the vertical direction prior to insertion. As explained previously, the orientation of the pump cassette housing 12 may utilize gravity to facilitate the flow of the surgical solution. In some implementations, the pump cassette housing 12 may be configured to be inserted into the cassette port 102 with one or more of the bridge tube 64, the pressure sensing module 70, and the identifier module 96 facing towards the surgical pump assembly 100. Because the pressure module 70 may face the surgical pump assembly 100, translation of the membranes 74A, 74B resulting from pressure variations may be into a depth of the cassette port 102 and generally may not interfere with the insertion and removal of the pump cassette 10. In this arrangement, the loading of the cassette may align the pressure sensing module 70 with a pressure sensor unit 104 of the control system 200.

As shown in FIG. 4, the cassette 10 may be inserted into the cassette port 102 along a docking path P_{d}, loading axis, and/or loading plane. In this manner, the primary plane Pₚ of the cassette 10 may generally be parallel to (e.g., aligned with the docking path P_{d}). Accordingly, the sensing module 70 may traverse an enclosed passage formed by the cassette port 102 and engage or abut the pressure sensing unit 104. For example, the translational engagement of the cassette 10 within the cassette port 102 along the docking path P_{d} may cause the membranes 74A, 74B of the pressure sensing unit 104 to abut, compress, or seat against the sensing surfaces of the pressure sensing unit 104. As shown, the membranes 74A, 74B and the sensing surfaces of the pressure sensing unit 104 may be arranged perpendicular to the docking path P_{d}. This arrangement may ensure that the sensing surfaces of the pressure sensing unit 104 (e.g., the surfaces aligned with the membranes 74A, 74B) are consistently and accurately positioned in contact with the membranes 74A, 74B.

In various implementations, the pressure sensing unit 104 may include one or more pressure sensing devices that may be oriented to detect the variations in the displacement of the membranes 74A, 74B. For example, the pressure sensing devices may include one or more fluidic pressure sensors or similar sensory devices that may detect the fluctuations in pressure in response to variations in electrical signals detected by one or more load cells, piezoresistive sensors, or similar devices. The accuracy and range of the sensor(s) may be designed to accommodate the application of the surgical pump assembly 100. In general, such pressure sensors may be capable of detecting a wide range of pressures from 1 mm Hg to 2000 mm Hg, and in many cases from 1 mm Hg to 1000 mm Hg. Though exemplary ranges are disclosed, it shall be understood that various types of sensors may be utilized to increase the range or accuracy of the pressure sensing unit 104.

Still referring to FIG. 4, the surgical pump assembly 100 may further include an identification sensor 106 located proximate the cassette port 102. In this manner, the identifier module 96 may be proximate and detectable by the identification sensor 106. In some implementations, the pressure sensing unit 104 may measure the displacement of the at least one flexible membrane 74A, 74B either remotely or via directed contact. For example, the pressure sensing unit 104 may measure the displacement of the at least one flexible membrane 74A, 74B via a detent that may be physically moved during the displacement. In some implementations, the pressure sensing unit 104 may measure the force acting upon the detent via the at least one flexible membrane 74A, 74B. The magnitude and/or force of displacement may then be converted into the quantified pressure value. The identification sensor 106 may likewise be capable of detecting the identifier module 96 remotely (e.g., with an RFID reader) or via physical interaction.

With reference now to FIG. 5, the control system 200 may effectuate the pump settings, detect and identify the type of pump cassette, and monitor (e.g., sample, detect, and/or otherwise obtain) the pressure within the pump cassette 10 during usage to ensure optimal settings are maintained. The control system 200 may include an electronic control unit (ECU) 202. The ECU 202 may include a processor 204 and a memory 206. The processor 204 may include any suitable processor 204 or any suitable number of processors, in addition to or other than the processor 204. In some implementations, the processor 204 may include one or more microprocessors, microcontrollers, application-specific integrated circuits (ASIC), or other circuitry configured to perform instructions, computations, and control various input/output signals to control the control system 200. The memory 206 may comprise a single disk or a plurality of disks (e.g., hard drives) and may include a storage management module that manages one or more partitions within the memory 206. In some implementations, memory 206 may include flash memory, semiconductor (solid-state) memory, or the like. The memory 206 may include Random Access Memory (RAM), a Read-Only Memory (ROM), Electrically Erasable Programmable Read-Only Memory (EEPROM), or a combination thereof. The memory 206 may include instructions that, when executed by the processor 204, cause the processor 204 to, at least, perform the functions associated with the components of the surgical pump assembly 100.

The memory 206 may, for example, include profile information 208 related to the number, type, and displacement profiles of the pressure sensing module 70. The profile information 208 may include a plurality of different profiles associated with different variations of the pump cassette 10. Each variation of the pump cassette 10 may be associated with different identifier data that may be readable by the identification sensor 106. In this manner, the various algorithms and methods related to converting displacement or force of the flexible membranes 74A, 74B into the quantified pressure value may be profiled and associated with different types of identification data. The memory 206 may further include pump parameter data 210, which may be associated with an expected, optimized, or otherwise desired amount of pressure associated with one or more flow rate settings of the surgical pump assembly 100. In addition, the memory may contain a filtering module 212 that compares the displacement or force from two or more pressure sensor modules 70 to compare values with expected values (e.g., in the pump parameter data 210), compare displacement and/or force values of each pressure sensor module 70 with each other, and/or the like. The filtering module 212 may average the values or remove certain values that are not within an expected value range. The values may be communicated to a user and/or automatically used by the control system 200 to maintain a desired flow rate, which may have been previously selected by the user and/or otherwise chosen and/or recommended to a user automatically by the control system 200.

According to some aspects of the disclosure, a pump cassette with pressure sensing is provided. The pump cassette includes a cassette housing having a first major wall and a second major wall spaced from the first major wall by a sidewall. An input aperture is defined by the cassette housing and configured to receive a surgical solution during a medical procedure. An output aperture is defined by the cassette housing and configured to expel the surgical solution during the medical procedure. A flow path is defined between the input aperture and the output aperture. The flow path includes a sensor segment having a sensor segment input opening, a sensor segment output opening, and a pressure module configured to react to changes in pressure. A sensor segment wall defines a portion of the flow path between the sensor segment input opening and the sensor segment output opening. The sensor segment wall includes a first sensor segment wall portion located between the sensor segment input opening and the sensor segment output opening and a second sensor wall portion extending transversely from the first sensor segment wall portion to a sensor segment wall terminal end. The flow path follows the sensor segment wall from the sensor segment input opening past the sensor segment wall terminal end and to the sensor segment output opening.

According to various aspects, the disclosure may implement one or more of the following features or configurations in various combinations:
the pressure module includes at least one flexible membrane is in fluid communication with the flow path proximate the sensor segment;
the at least one flexible membrane is configured to react by at least one of translating, moving, deflecting, flexing, or deforming in response to changes in the pressure of the surgical solution;
the reaction of the at least one flexible membrane is configured to be converted into a quantified pressure value;
the at least one flexible membrane includes a first flexible membrane and a second flexible membrane;
the at least one flexible membrane deflects in response to variations in the in the pressure of the surgical solution and a magnitude of the deflection is detectable by a sensor configured to abut the membrane;
the sensor segment is bounded by the sidewall and a sensor cap coupled to the sidewall, and the at least one flexible membrane is coupled to the sensor cap opposite the sensor segment of the flow path;
the sensor cap includes at least one cap opening aligned with the at least one flexible membrane and exposing the at least one flexible membrane to surgical solution along the flow path between the sensor segment input opening and the sensor segment output opening;
the at least one cap opening is located in the flow path proximate the second sensor wall portion before the sensor segment wall terminal end;
the flow path within the sensor segment comprises a plurality of sensor segment route sections including a first sensor segment route section that extends from the sensor segment input opening, past the second sensor segment wall portion, and to a second sensor segment route section;
the flow path within the sensor segment comprises a plurality of sensor segment route sections including a first route section that extends opposite a second route section on an opposite side of the second sensor segment wall portion;
the second sensor segment route section extends transversely from the first sensor segment route section, around the sensor segment wall terminal end, to a third sensor segment route section;
the third sensor segment route section extends in an opposite direction from the first sensor segment route section on an opposite side of the second sensor segment wall portion to a fourth sensor segment route section;
the fourth sensor segment route section extends opposite the second sensor segment route section, along the first sensor segment wall portion to the sensor segment output opening;
the sensor segment input opening and the sensor segment output opening abut opposite sides of the first sensor segment wall portion
the at least one flexible membrane is integrally formed of a silicone material;
a face portion of the at least one flexible membrane is less than 550 μm thick;
the sidewall includes a first lateral sidewall section and a second lateral sidewall section spaced by a first longitudinal sidewall section and a second longitudinal sidewall section, the second longitudinal sidewall section defining a pair of opposing sections that extend to a bay that extends towards the first longitudinal sidewall section;
the sensor segment is located on one of the opposing sections of the second longitudinal sidewall section proximate the bay;
a bridge tube is coupled to the opposing sections and is bowed towards the first longitudinal sidewall section within the bay, the bridge tube defining a segment of the flow path;
the bridge tube is formed of a flexible material and configured to be engaged by a peristaltic pump; and
a bridge tube is coupled to the opposing sections and is bowed towards the first longitudinal sidewall section within the bay, the bridge tube defining a segment of the flow path and configured to be engaged by a peristaltic pump.

According to another aspect of the disclosure, a pump cassette with pulsation abatement is provided. The pump cassette includes a cassette housing having a first major wall and a second major wall spaced from the first major wall by a sidewall. The sidewall includes a first lateral sidewall section and a second lateral sidewall section spaced by a first longitudinal sidewall section and a second longitudinal sidewall section. An input aperture is defined by the cassette housing and configured to receive a surgical solution during a medical procedure. An output aperture is defined by the cassette housing and configured to expel the surgical solution during the medical procedure. A flow path is defined between the input aperture and the output aperture. The flow path includes an input flow path segment extending from the input aperture and at least partially along the first lateral sidewall section to one of the opposing sections of the second longitudinal sidewall section. The flow path further includes an output flow path segment extending at least partially along the second lateral sidewall section to the output aperture. A flow path branch is between the input aperture and the output aperture that extends to a cavity within the cassette housing for pulse abatement of a surgical solution.

According to yet another aspect of the disclosure, a pump cassette with pulsation abatement is provided. The pump cassette includes a cassette housing having a first major wall and a second major wall spaced from the first major wall by a sidewall. The sidewall includes a first lateral sidewall section and a second lateral sidewall section spaced by a first longitudinal sidewall section and a second longitudinal sidewall section. An input aperture is defined by the cassette housing and configured to receive a surgical solution during a medical procedure. An output aperture is defined by the cassette housing and configured to expel the surgical solution during the medical procedure. A flow path is defined between the input aperture and the output aperture. The flow path includes an input flow path segment extending from the input aperture and at least partially along the first lateral sidewall section to the second longitudinal sidewall section. The flow path further includes an output flow path segment extending at least partially along the second lateral sidewall section to the output aperture. A flow path branch is between the input aperture and the output aperture that extends to a cavity within the cassette housing for pulse abatement of a surgical solution.

According to another aspect of the disclosure, a pump cassette with pulsation abatement is provided. The pump cassette includes a cassette housing having a first major wall and a second major wall spaced from the first major wall by a sidewall. The sidewall includes a first lateral sidewall section and a second lateral sidewall section spaced by a first longitudinal sidewall section and a second longitudinal sidewall section. An input aperture is defined by the cassette housing and configured to receive a surgical solution during a medical procedure. An output aperture is defined by the cassette housing and configured to expel the surgical solution during the medical procedure. A flow path is defined between the input aperture and the output aperture. The flow path includes an input flow path segment extending from the input aperture and at least partially along the first lateral sidewall section to one of the opposing sections of the second longitudinal sidewall section. The flow path further includes an output flow path segment extending at least partially along the second lateral sidewall section to the output aperture. A flow path branch is between the input aperture and the output aperture that extends to a cavity within the cassette housing for pulse abatement of a surgical solution.

According to various aspects, the disclosure may implement one or more of the following features or configurations in various combinations:
the pulsation abatement flow path branch extends at least partially along the second lateral sidewall section in a direction opposite the output flow path segment;
the flow path proximate the pulsation abatement flow path branch comprises a plurality of route sections including a first route section extending along the output flow path segment to a second route section that extends perpendicular to the first route section;
the second route section extends to a juncture between a third route section defined by the pulsation abatement flow path branch and a fourth route section extending towards the output aperture;
the third route section extends in an opposite direction from the first route section;
the fourth route section extends to a fifth route section that extends transversely from the fourth route section to the output aperture;
the air pocket is hermetically sealed within the cavity and pressurized;
the second longitudinal sidewall section includes a pair of opposing sections extending to a bay that extends towards the first longitudinal sidewall section;
an intermediary flow path segment extends from the input flow path segment to the output flow path segment, the intermediary flow path segment including a bridge tube coupled to the opposing sections of the second longitudinal sidewall section and is bowed towards the first longitudinal sidewall section within the bay;
the bridge tube is formed of a flexible material and configured to be engaged by a peristaltic pump;
the first lateral sidewall section and the second lateral sidewall section are, at least in part, planar and parallel;
the first longitudinal sidewall section is perpendicular to the first lateral sidewall section and the second lateral sidewall section;
the flow path further includes a sensor segment including a sensor segment input opening, a sensor segment output opening, and a pressure module configured to react to changes in pressure;
at least part of the flow path between the sensor segment input opening and the sensor segment output opening is offset with respect to a direction between the first major wall and the second major wall from the input flow path segment and the output flow path segment;
at least part of the flow path is offset with respect to a direction between the first major wall and the second major wall from the input flow path segment and the output flow path segment;
the first major wall and the second major wall are separately formed and coupled to one another;
the first major wall includes a pair of parallel first ribs defining a channel therebetween and the second major wall includes a second rib located in the channel;
the pulsation abatement flow path branch extends at least partially along the first longitudinal sidewall section in a direction upstream from the output aperture;
the flow path further includes a sensor segment configured to react to changes in pressure located between the input aperture and the output aperture, and wherein the pulsation abatement flow path branch is located downstream of the sensor segment.
the pulsation abatement flow path branch includes at least one switchback;
the pulsation abatement flow path branch is at least partially aligned with a sensor segment input opening;
the pulsation abatement flow path branch includes a narrowing cross-section from the flow path;
the pulsation abatement flow path branch is located upstream from a sensor segment;
the pulsation abatement flow path branch is located downstream from a sensor segment;
the pulsation abatement flow path branch extends at an acute angle from the flow path;
the flow path further includes a sensor segment configured to react to changes in pressure located between the input aperture and the output aperture, and wherein the pulsation abatement flow path branch is located upstream of the sensor segment; and
the pulsation abatement flow path branch includes at least two switchbacks.

According to yet another aspect of the disclosure, a pump cassette with pressure sensing is provided. The pump cassette includes a cassette housing having a first major wall and a second major wall spaced from the first major wall by a sidewall. An input aperture is defined by the cassette housing and configured to receive a surgical solution during a medical procedure. An output aperture is defined by the cassette housing and configured to expel the surgical solution during the medical procedure. A flow path is defined between the input aperture and the output aperture and includes a sensor segment. At least two flexible membranes are coupled to the sensor segment and in fluid communication with the flow path.

According to various aspects, the disclosure may implement one or more of the following features or configurations in various combinations:
the at least two flexible membranes are each configured to react response to changes in the pressure of the surgical solution;
the reaction of the at least two flexible membranes are configured to be converted into a quantified pressure value;
the reaction of the at least two flexible membranes are configured to be converted into a quantified pressure value and compared;
the cassette housing is configured to be loaded into a loading dock of a surgical pump assembly along a loading axis and the at least two flexible membranes are configured to expand and contract in a direction parallel to the loading axis; and
the cassette housing defines a primary plane between the first and second major walls and the at least two flexible membranes are configured to expand and contract in a direction parallel to primary plane.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

As used herein, the terms “about” and “approximate” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

1. A pump cassette with pressure sensing, the pump cassette comprising:
a cassette housing comprising a first major wall and a second major wall spaced from the first major wall by a sidewall;
an input aperture defined by the cassette housing and configured to receive a surgical solution during a medical procedure;
an output aperture defined by the cassette housing and configured to expel the surgical solution during the medical procedure; and
a flow path defined between the input aperture and the output aperture, the flow path comprising:
a sensor segment including a sensor segment input opening, a sensor segment output opening, and a pressure module configured to react to changes in pressure of the surgical solution; and
a sensor segment wall defining a portion of the flow path between the sensor segment input opening and the sensor segment output opening, the sensor segment wall including a first sensor segment wall portion located between the sensor segment input opening and the sensor segment output opening and a second sensor wall portion extending transversely from the first sensor segment wall portion to a sensor segment wall terminal end, wherein the flow path follows the sensor segment wall from the sensor segment input opening past the sensor segment wall terminal end and to the sensor segment output opening.

2. The pump cassette of claim 1, wherein the pressure module includes at least one flexible membrane in fluid communication with the flow path proximate the sensor segment.

3. The pump cassette of claim 2, wherein the at least one flexible membrane is configured to react by at least one of translating, moving, deflecting, flexing, or deforming in response to changes in the pressure of the surgical solution.

4. The pump cassette of claim 2, wherein the sensor segment is bounded by the sidewall and a sensor cap coupled to the sidewall, and the at least one flexible membrane coupled to the sensor cap opposite the sensor segment of the flow path.

5. The pump cassette of claim 4, wherein the sensor cap includes at least one cap opening aligned with the at least one flexible membrane and exposing the at least one flexible membrane to surgical solution along the flow path between the sensor segment input opening and the sensor segment output opening.

6. The pump cassette of claim 1, wherein the flow path within the sensor segment comprises a plurality of sensor segment route sections including a first route section that extends opposite a second route section on an opposite side of the second sensor segment wall portion.

7. The pump cassette of claim 1, wherein the sidewall includes a first lateral sidewall section and a second lateral sidewall section spaced by a first longitudinal sidewall section and a second longitudinal sidewall section, the second longitudinal sidewall section defining a pair of opposing sections that extend to a bay that extends towards the first longitudinal sidewall section.

8. The pump cassette of claim 7, wherein the sensor segment is located on one of the opposing sections of the second longitudinal sidewall section proximate the bay.

9. The pump cassette of claim 8, wherein a bridge tube is coupled to the opposing sections and is bowed towards the first longitudinal sidewall section within the bay, the bridge tube defining a segment of the flow path and configured to be engaged by a peristaltic pump.

10. A pump cassette with pulsation abatement, the pump cassette comprising:
a cassette housing including a first major wall and a second major wall spaced from the first major wall by a sidewall;
the sidewall including a first lateral sidewall section and a second lateral sidewall section spaced by a first longitudinal sidewall section and a second longitudinal sidewall section;
an input aperture is defined by the cassette housing and configured to receive a surgical solution during a medical procedure;
an output aperture defined by the cassette housing and configured to expel the surgical solution during the medical procedure; and
a flow path traveling upstream between the input aperture and the output aperture, the flow path comprising:
an input flow path segment extending from the input aperture and at least partially along the first lateral sidewall section to the second longitudinal sidewall section;
an output flow path segment extending at least partially along the second lateral sidewall section to the output aperture; and
a flow path branch between the input aperture and the output aperture that extends to a cavity within the cassette housing for pulse abatement of a surgical solution.

11. The pump cassette of claim 10, wherein the pulsation abatement flow path branch extends at least partially along the second lateral sidewall section in a direction opposite the output flow path segment.

12. The pump cassette of claim 10, wherein the flow path proximate the pulsation abatement flow path branch comprises a plurality of route sections including a first route section extending along the output flow path segment to a second route section that extends perpendicular to the first route section.

13. The pump cassette of claim 12, wherein the second route section extends to a juncture between a third route section defined by the pulsation abatement flow path branch and a fourth route section extending towards the output aperture.

14. The pump cassette of claim 10, wherein an air pocket is hermetically sealed within the cavity and pressurized.

15. The pump cassette of claim 10, wherein at least part of the flow path is offset with respect to a direction between the first major wall and the second major wall from the input flow path segment and the output flow path segment.

16. The pump cassette of claim 10, wherein the pulsation abatement flow path branch includes at least one switchback.

17. The pump cassette of claim 16, wherein the pulsation abatement flow path branch is located upstream from a sensor segment.

18. The pump cassette of claim 16, wherein the pulsation abatement flow path branch is located downstream from a sensor segment.

19. A pump cassette with pressure sensing, the pump cassette comprising:
a cassette housing including a first major wall and a second major wall spaced from the first major wall by a sidewall;
an input aperture is defined by the cassette housing and configured to receive a surgical solution during a medical procedure;
an output aperture is defined by the cassette housing and configured to expel the surgical solution during the medical procedure;
a flow path defined between the input aperture and the output aperture and including a sensor segment; and
at least two flexible membranes coupled to the sensor segment and in fluid communication with the flow path.

20. The pump cassette of claim 19, wherein a reaction of the at least two flexible membranes are configured to be converted into a quantified pressure value and compared.
